(19) United States
(12) Patent Application Publication
(10) Pub. No.: US 2015/0011957 A1
(43) Pub. Date: Jan. 8, 2015

(54) Nightwear absorbent, washable and reusable, and its manufacturing process

(71) Applicants: Pascal Cloutier, Coaticook (CA); Kathleen Trudeau, Coaticook (CA); Marc Roy, Sherbrooke (CA)

(72) Inventors: Pascal Cloutier, Coaticook (CA); Kathleen Trudeau, Coaticook (CA); Marc Roy, Sherbrooke (CA)

(21) Appl. No.: 13/987,165

(22) Filed: Jul. 8, 2013

(51) Int. Cl. A61F 13/49

(52) U.S. Cl. CPC A61F 13/49006
USPC 604/377

(57) ABSTRACT

The present invention is a nightwear for children which is absorbent, washable, and reusable, and which is constituted of inner portion formed of a super light and soft fabric made of micropolar allowing to keep the body dry due to the fact that urine is directly absorbed by a cotton fabric forming a central portion, and which is connected to an outer portion made of waterproof fabric being provided with horizontal seams dive to allow urine to move towards the seams connecting the central portion to the inner portion of the nightwear. A seam sealer is used to prevent that urine passes through holes in the seams. The garment has elastic band and Velcro strips at the waist for keeping urine inside the nightwear. The nightwear has elastic band at thighs for the short pant and at ankles for the long pant or the babygro for keeping urine inside the nightwear.

### BACKGROUND OF THE INVENTION

1. Field of the Invention

The invention concerns the garments. More particularly, the invention is directed to a nightwear for children which is absorbent, washable and reusable.

2. Description of the Related Art

A search of prior art records has unveiled the following patents:
U.S. Pat. No. 3,648,699 issued in 1972 to Anderson et al.
U.S. Pat. No. 3,180,336 issued in 1965 to Bett et al.
U.S. Pat. No. 2,621,336 issued in 1952 to Wendroff.
U.S. Pat. No. 4,114,621 issued in 1978 to Mims, Jr.
U.S. Pat. No. 7,074,215 issued in 2006 to Ashton et al.
U.S. Pat. No. 5,770,529 issued in 1998, to Dennis et al.
U.S. Pat. No. 6,245,051 issued in 2001, to Zenker et al.
U.S. Pat. No. 6,573,424 issued in 2003, to Raidel et al.
U.S. Pat. No. 6,965,058 issued in 2005 to Raidel et al.
U.S. Pat. No. 7,145, 054 issued in 2006 to Zander et al.
U.S. Pat. No. 6,585,840 issued in 2003 to Rabe et al.
U.S. Pat. No. 4,886,512 issued in 1989 to Damico et al.
U.S. Pat. No. 6,487,727 issued in 2002 to Harsant.
U.S. Pat. No. 8,394,074 issued in 2013 to Piette et al.
U.S. Pat. No. 7,077,833 issued in 2006 to Bonham.
U.S. Pat. No. 6,413,249 issued in 2002 to Turi et al.
U.S. Pat. No. 5,527,303 issued in 1996 to Milby Jr. et al.
U.S. Pat. No. 5,445,628 issued in 1995 to Gipson et al.
U.S. Pat. No. 5,380,310 issued in 1995 to Mitrani.

3. Advantages

Economic and ecological environment;
no need to use the disposable diapers which are harmful for the environment;
no need to wash the bed sheets and blankets;
to prevent any irritation of the skin;
allows to increase the self-confidence;
allows to preserve the mattresses, and the padded armchairs and chairs;
and more need to use a cushioning which moves under the child during the night.

### SUMMARY OF THE INVENTION

According to the present invention, it is described an absorbent nightwear, washable and reusable which is suitable for children. This nightwear consists of an inner portion formed of a super light and soft fabric made of micropolar allowing to keep the body dry due to the fact that urine is directly absorbed by a cotton fabric forming a central portion, and which is connected to an outer portion made of waterproof fabric also provided with horizontal seams dive to allow urine to move towards the seams connecting the central portion with the inner portion of the nightwear, and a seam sealer is used to prevent that urine passes through holes in the seams.

The nightwear has elastic band and Velcro strips at the waist for keeping urine inside the nightwear.

The nightwear has elastic band at thighs for the short pant and to ankles for the long pant or the babygro in order to keep urine inside the nightwear.

The foregoing and other objects, features, and advantages of this invention will become more readily apparent from the following detailed description of a preferred embodiment with reference to the accompanying drawings, wherein the preferred embodiment of the invention is shown and described, by way of examples. As will be realized, the invention is capable of other and different embodiments, and its several details are capable of modifications in various obvious respects, all without departing from the invention.

Accordingly, the drawings and description are to be regarded as illustrative in nature, and not as restrictive.

### BRIEF DESCRIPTION OF THE DRAWINGS

The accompanying drawings show illustrative embodiment of the present invention from which the features and advantages will be readily apparent.

FIG. 1 shows a view of a long pant of nightwear.

FIG. 2 shows a view of a short pant of nightwear.

FIG. 3 shows a view of a babygro.

FIG. 4 shows a view of a sectional view of the nightwear.

FIG. 5 shows an exploded view of the nightwear.

### DETAILED DESCRIPTION OF THE ILLUSTRATED EMBODIMENT

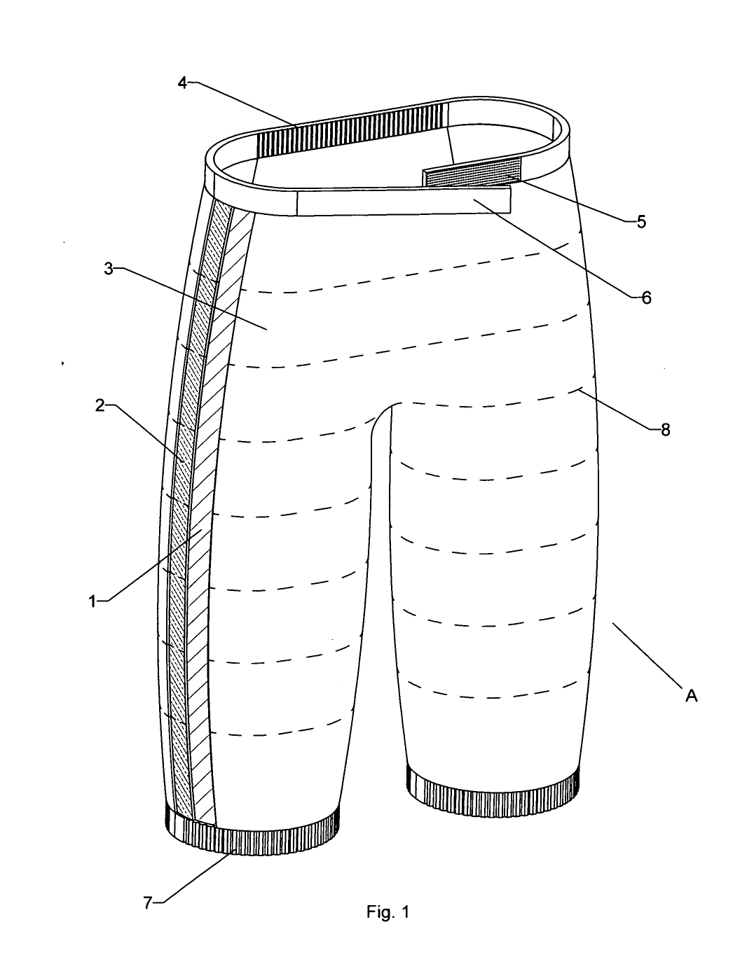

Referring now to the drawings, more particularly to FIG. 1, on see a long pant (A) of the nightwear for children which is absorbent, washable and reusable, and which is constituted of an inner portion formed of a super light and soft fabric made of micropolar allowing to keep the body dry due to the fact that urine is directly absorbed by a cotton fabric forming a central portion (2), and which is joined to an outer portion (3) formed from a waterproof fabric made of polyester.

The nightwear is also provided with horizontal seams dive (8) to allow the urine to be directed towards the seams connecting the central portion (2) to the inner portion (1), and a seam sealer is used in order to prevent that the urine passes through holes in the seams.

The nightwear is provided to the waist of elastic band (4) and Velcro strips (5)(6) or rib knit (not shown) for keeping urine inside the nightwear.

The nightwear has elastic band (7) at thighs for the short pant and to ankles for the long pant or babygro in order to keep urine inside the nightwear.

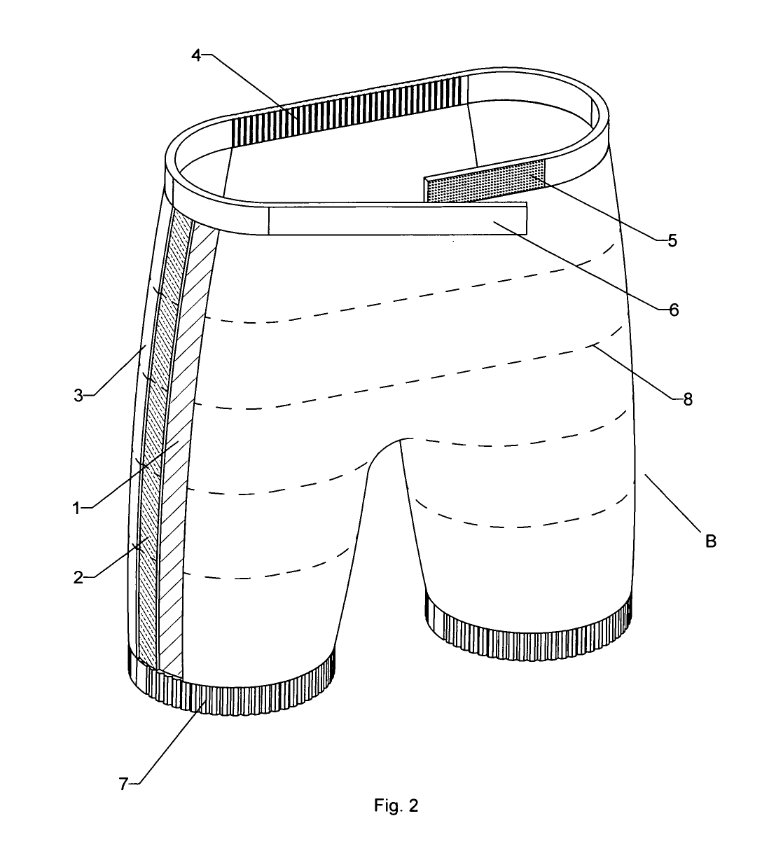

As shown in FIG. 2, one see a short pant (B) of the nightwear for children, which is absorbent, washable and reusable, and which is constituted of an inner portion (1) formed of a super light and soft fabric made of micropolar allowing to keep the body dry due to the fact that urine is directly absorbed by a cotton fabric forming a central portion (2), and which is joined to an outer portion (3) formed from a waterproof fabric made of polyester.

The nightwear is also provided with horizontal seams dive (8) to allow the urine to be directed towards the seams connecting the central portion (2) to the inner portion (1), and a seam sealer is used in order to prevent that the urine passes through holes in the seams.

The nightwear is provided to the waist of elastic band (4) and Velcro strips (5)(6) or rib knit (not shown) for keeping urine inside the nightwear.

The nightwear has elastic band (7) at thighs for the short pant and to ankles for the long pant or bodygro for keeping urine inside the nightwear.

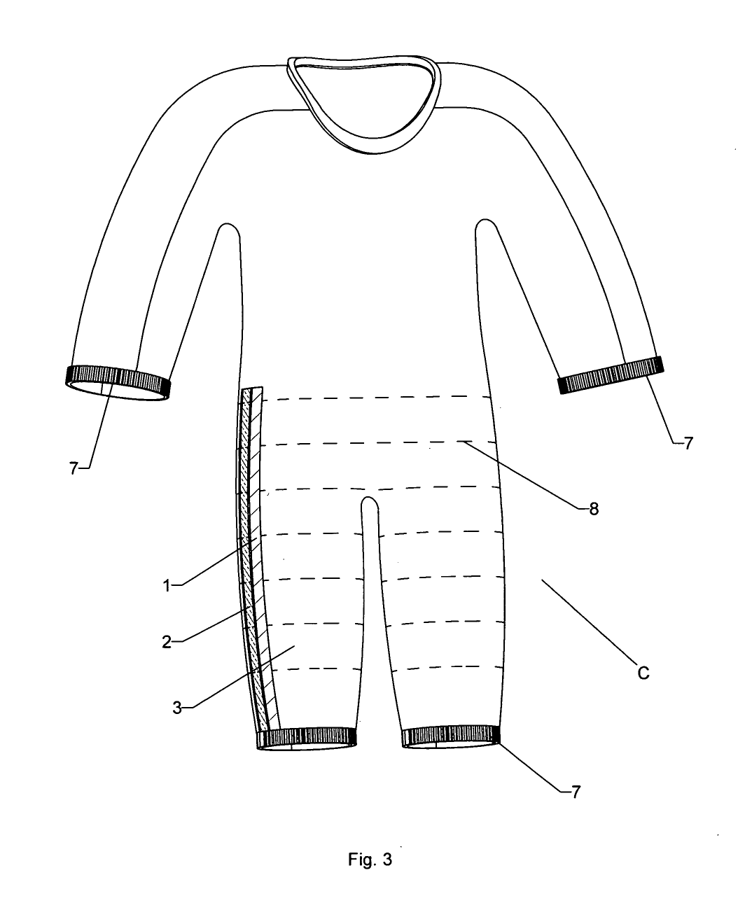

As shown in FIG. 3, one see a badygro (C) for children, which is absorbent, washable and reusable, and which is constituted of an inner portion (1) formed of a super light and soft fabric made of micropolar allowing to keep the body dry due to the fact that urine is directly absorbed by a cotton fabric forming a central portion (2), and which is joined to an outer portion (3) formed from a waterproof fabric made of polyester.

The badygro is also provided of with horizontal seams dive (8) to allow the urine to be directed towards the seams connecting the central portion (2) to the inner portion (1), and a seam sealer is used in order to prevent that the urine passes through holes in the seams. The babygro has elastic band (7) to arms and ankles or rib knit (not shown) for keeping the urine inside the nightwear.

Push buttons or Velcro strips (not shown) are generally used to close the front and upper portion of the babygro.

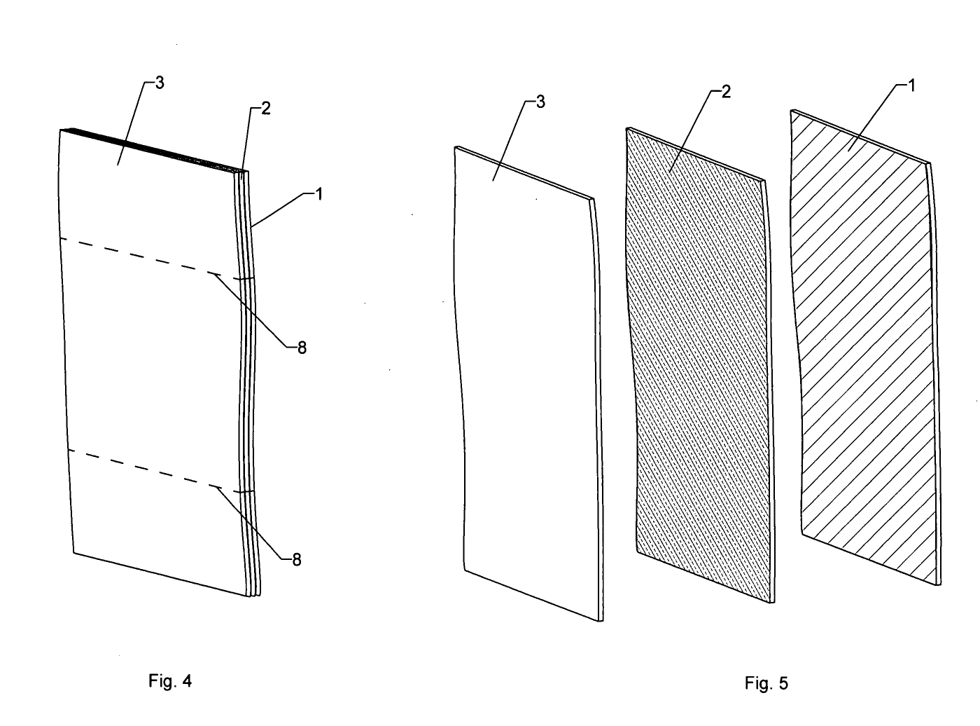

As shown in FIG. 4, one see a sectional view of the nightwear.

As shown in FIG. 5, one see an exploded view of the nightwear.

The nightwear can reach several categories of people in the world population, including children, elderly, sick or people disabilities, and all people suffering from incontinence night or bedwetting.

Although only a single embodiment of the present invention has been described and illustrated, the present invention is not limited to the features of this embodiment, but includes all variations and modifications within the scope of claims.

I claim:

1. A nightwear for children, which is absorbent, washable and reusable, and which comprises an inner portion formed of a super light and soft fabric made of micropolar allowing to keep the body dry due to the fact that urine is directly absorbed by a cotton fabric forming a central portion, and which is connected to an outer portion constituted of waterproof fabric made of polyester and being provided with horizontal seams dive to allow urine to move towards the seams connecting the central portion to the inner portion of the nightwear, and a seam sealer is used to prevent urine passes through holes in the seams.

2. The nightwear as claimed in claim 1 includes elastic band and Velcro strips at the waist for keeping urine inside the nightwear.

3. The nightwear as claimed in claim 1 is provided of elastic band at thighs for the short pant for keeping urine inside the nightwear.

4. The nightwear as claimed in claim 1 is provided of elastic band at ankles for the long pant and for the babygro for keeping urine inside the nightwear.

5. The nightwear as claimed in claim 1 characterized in that it is also adapted for the elderly, sick and disabled persons, and for the persons suffering from incontinence night or bedwetting.
